(19) United States
(12) Patent Application Publication
(10) Pub. No.: US 2013/0096163 A1
(43) Pub. Date: Apr. 18, 2013

(54) Injectable dosage form of flupirtine

(71) Applicants: Christoph Martin Hoock, Dresden (DE); Asal Qadan, Dresden (DE); Bernd Terhaag, Dresden (DE); Rudy Thoma, Michendorf (DE)

(72) Inventors: Christoph Martin Hoock, Dresden (DE); Asal Qadan, Dresden (DE); Bernd Terhaag, Dresden (DE); Rudy Thoma, Michendorf (DE)

(73) Assignee: AWD.PHARMA GMBH & CO. KG, Radebeul (DE)

(21) Appl. No.: 13/704,277

(22) PCT Filed: Jun. 14, 2011
(86) PCT No.: PCT/EP2011/059863

§ 371 Date: Dec. 14, 2012

(30) Foreign Application Priority Data
Jun. 14, 2010 (DE) 10 2010 030 053.5

(51) Int. Cl. A61K 31/44

(52) U.S. Cl. CPC A61K 31/44
USPC 514/353

(57) ABSTRACT

The present invention relates to a flupirtine-containing lyophilisate, the use of this lyophilisate to produce a pharmaceutical composition to be parenterally applied, a procedure to produce a flupirtine-containing pharmaceutical composition to be parenterally applied, and a procedure to produce the flupirtine-containing lyophilisate as well as the flupirtine-containing pharmaceutical composition produced using the lyophilisate. For this purpose a lyophilisate is provided which contains the active ingredient flupirtine in the form of a physiologically tolerated salt, which has a solubility in water of at least 2.5 mg/ml, preferably at least 5 mg/ml, especially preferably at least 10 mg/ml, and contains one or more cyclodextrins or cyclodextrin derivatives, and which may be used to produce a pharmaceutical composition to be parenterally applied.

The present invention relates to a flupirtine-containing lyophilisate, the use of this lyophilisate to produce a pharmaceutical composition to be parenterally applied, a procedure to produce a flupirtine-containing pharmaceutical composition to be parenterally applied, and a procedure to produce the flupirtine-containing lyophilisate as well as the flupirtine-containing pharmaceutical composition produced using the lyophilisate.

Flupirtine (Katadolon®; 2-amino-3-carbethoxyamino-6-(4-fluorbenzylamino)-pyridine=2-amino-3-ethoxycarbonylamino-6-(p-fluorbenzylamino)-pyridine) is a centrally acting, non-opioid analgesic. For many years, flupirtine respectively its physiologically tolerated salts have been successfully used in the therapy of e.g. neuralgias, pain due to degenerative joint diseases, headaches and postoperative pain. According to DE 41 22 166 A1, flupirtine can also be used as a medication to treat disorders or disorder symptoms due to muscle tension or resulting from such muscle tension. In DE 43 27 516 the use of flupirtine in the treatment of cerebral ischaemia and neurodegenerative diseases is further described. From DE 195 41 405 A1, the use of flupirtine in the prophylaxis and therapy of diseases that are accompanied by an impaired haematopoetic cell system is known. Furthermore, DE 100 48 969 A1describes the use of flupirtine in the treatment of tinnitus. The production of flupirtine and its physiologically usable salts is described in DE 17 95 858 C2, DE 31 33 519 C2, and DE 34 16 609 A1.

Flupirtine is mainly applied orally. Accordingly, DE 93 21 574 U1 describes, for example, pharmaceutical formulations in the form of tablets, granules or pellets containing flupirtine maleate as an active ingredient. From DE 43 19 649 A1, solid flupirtine-containing oral dosage forms with controlled release of the active ingredient are known.

Due to the good analgesic effect of flupirtine it is desirable to apply flupirtine parenterally, to achieve a rapid effect. This contrasts with the fact that flupirtine and its physiologically tolerated salts are insoluble, or scarcely soluble, in aqueous solutions, and in most organic solvents which are physiologically tolerated.

The patent application DE 34 16 609 A1 describes pharmaceutical formulations in the form of injectable flupirtine-gluconate-solutions produced using suitable solvents. As solvent, especially a mixture of polyethylene glycol and water, or a mixture of glycofurol and water is used. However, the described injection solutions show a number of serious disadvantages, in so far as the flupirtlne-gluconate-solutions produced using mixtures of polyethylene glycol and water or glycofurol and water are very hypertonic, and therefore only suitable for intramuscular application. Due to the very acidic solution and the excipients used, such as sodium disulfite and propylene glycol, injections often result in irritations at the site of application. In addition, the described flupirtine-gluconate-solutions do not have sufficient physical stability. They are stable only for a limited period, so that after only a few weeks a process of precipitation may begin which markedly limits the shelf life of the preparation. It has been found that the physical stability of the flupirtine solutions also depends to a high degree on storage temperature. As the precipitation starts markedly earlier at lower temperatures, it is necessary to maintain a minimum temperature of 20° C. for storage of the flupirtine solutions to improve their stability. Ideally, ampoules containing such injection solutions should be stored at temperatures of 25° C. to 30° C. which, however, can hardly be achieved in practice.

WO 2004/112754 A1 discloses a lyophilisate containing the active ingredient flupirtine in its base form or as a physiologically tolerated salt, which may be used to produce a pharmaceutical composition to be parenterally applied. As the only exemplary embodiment, flupirtine gluconate was lyophilized. In contrast to common flupirtine injection solutions, in which it is not possible to use purely aqueous media but only solvent systems containing a high proportion of organic solvents such as propylene glycol, the disclosed flupirtine lyophilisates are sufficiently soluble in aqueous systems. In addition, the flupirtine lyophltisates have the advantage that heating is not necessary when dissolving the lyophilisate since the disclosed flupirtine lyophilisates quickly dissolve even at room temperature. The reconstitution of the flupirtine lyophilisate in purely aqueous media yields clear flupirtine solutions which are stable over several hours and do not show precipitation processes. However, the shelf life of the described lyophilisates at room temperature is limited. After only a few weeks or months at room temperature the lyophilisate solidifies. This can be observed visually and results in impaired solubility. The reason identified is the rearrangement of the gluconic acid into its lactone form, accompanied by a release of water.

The task underlying this invention consists in providing means and procedures to produce flupirtine-containing pharmaceutical compositions that are suitable for parenteral application and do not show the disadvantages of parenteral pharmaceutical compositions known in the state of the art: that is, especially when injected, they have no side effects such as irritations, and in additon they show an improved physical and chemical stability so that they may be stored at room temperature for an extended period.

The present invention solves its underlying technical problem by providing a lyophilisate which contains the active ingredient flupirtine in the form of a physiologically tolerated salt having a solubility in water of at least 2.5 mg/ml, preferably at least 5 mg/ml, more preferably at least 10 mg/ml, and which contains one or more cyclodextrins or cyclodextrin derivatives, and which may be used to produce a pharmaceutical composition to be parenterally applied.

The reconstitution of the flupirtine lyophilisate according to the invention yields very clear flupirtine solutions that are stable over several hours and do not show any precipitation processes. Due to their stability, the flupirtine solutions produced using flupirtine lyophilisates according to to the invention are outstandingly suitable for injection, especially in the form of injection solutions or infusion solutions. To produce liquid dosage forms from the flupirtine lyophilisates according to to the invention purely aqueous media are suitable. In addition, the flupirtine lyophilisates according to the invention have the advantage that it is not necessary to heat the lyophilisate when dissolving it, since the flupirtine lyophilisates according to the invention already dissolve quickly at room temperature.

Moreover and surprisingly, it has been found that the disadvantages and problems known in the state of the art in handling and storage of a flupirtine lyophilisate intended to produce liquid formulations for injection may be completely remedied by using the lyophilisate according to the invention. The flupirtine lyophilisates according to to the invention have the advantage of remaining stable over many months without losing their ability to be reconstituted in aqueous media. Moreover, in the lyophilisates according to to the invention, the use of both antioxidants and detergents can be dispensed with advantageously, by using cyclodextrins or cyclodextrin derivatives, without impairing stability or shelf life of either the lyophilisate or the injection solution produced from it, compared to the known preparations.

Hypertonic solutions may lead to haemolysis when administered quickly in undiluted solution, e.g. by injection. However, the compositions according to the invention have the advantage that they result in a nearly isotonic solution, even when reconstituted in a comparatively small volume of liquid. Added to their better tolerated pH value of 3.0 to 3.7, the consequence of this is that the solutions produced from the flupirtine lyophilisates according to to the invention may be injected not only intramuscularly, but also intravenously. The common dosage unit of 100 mg of flupirtine (the quantity given relates to the flupirtine base) may be advantageously reconstituted in this form of preparation with only 3 ml of water.

The lyophilisates according to to the invention can be reconstituted and/or diluted as desired. Accordingly, the flupirtine lyophilisate according to the invention may be used optionally to produce intramuscular or intravenous injection solutions, and also to prepare infusion solutions. The reconstituted aqueous preparation may also be used as an admixture to commonly used infusion solutions. This form of use is, above all, advantageous for those patients who require systemic pain treatment in combination with other therapeutic measures. Since the lyophilisates according to to the invention characteristically dissolve very quickly, they can be reconstituted directly before application.

In the context of the present invention “lyophilisate” means a material which is obtained by drying it, in its frozen form, in a vacuum by sublimation of its solvent. A freeze-dried material obtained in this manner is very porous and maintains its original volume. In the context of the present invention, “pharmaceutical composition” or “pharmaceutical preparation” means a mixture which is used for diagnostic, therapeutic and/or prophylactic purposes, i.e. which promotes or reconstitutes the health of the human or animal body, and which contains at least one natural or synthetically produced active ingredient which causes the therapeutic effect.

The pharmaceutical composition may include commonly used additives in the specialist field, such as stabilizers, matrix forming agents, or other substances used to produce pharmaceutical compositions, especially to produce liquid dosage forms.

In the lyophilisate according to the invention, flupirtine is present in the form of a physiologically tolerated salt that has a solubilty in water of at least 2.5 mg/ml. Preferably the solubility of the flupirtine salt used in the lyophilisate according to the invention is at least 5 mg/ml, more preferably at least 10 mg/ml.

The solubility of the salt used is determined by stirring a suspension of a defined quantity of the corresponding salt for 30 minutes in a closed container in fully desalinated water at 25° C. The undissolved portion of the salt is filtered off, the filtrate is diluted to a suitable concentration range, and the flupirtine concentration of the solution determined using HPLC-UV (high performance liquid chromatography with UV-detection).

In a preferred embodiment of the invention, one dosage unit of the lyophilisate contains 50 to 250 mg of flupirtine; this quantity relates to the flupirtine base. More preferably, the lyophilisate contains 80 to 120 mg, and most preferably 100 mg of flupirtine, relating to the free flupirtine base.

It is appropriate that the flupirtine salt used is sufficiently stable to ensure a satisfactory shelf life of the lyophilisate. For instance, the stability of the flupirtine salt intended for use may be determined by its HPLC purity, where the decrease of the flupirtine content after 100 days at 25° C. and 60 % rel. humidity should be less than 0.05 % (a suitable HPLC method is described in PCT/US2009/046775). It is further appropriate that the flupirtine salt used should be inert towards the cyclodextrins and/or cyclodextrin derivatives used in the lyophilisate according to the invention, and, if applicable, inert towards the other components of the lyophilisate.

In a preferred embodiment of the invention, the flupirtine salt contained in the lyophilisate is selected from flupirtine mesilate, flupirtine besilate and/or flupirtine phosphate. Table 1shows the respective solubility of each of these flupirtine salts in water at 25° C. The solubility has been determined as described above.

TABLE 1

| Flupirtine salt | Solubility (mg/ml) |
|---|---|
| Flupirtine mesilate | 16.8 |
| Flupirtine besilate | 2.8 |
| Flupirtine phosphate | 13.6 |

Most preferably the lyophilisate according to the invention contains flupirtine mesilate as its physiologically tolerated flupirtine salt. According to the invention it is further intended that the lyophilisate contains the acidic component of the flupirtine mesilate, i.e. methanesulfonic acid, in a quantity of 10 mg to 100 mg, preferably 20 mg to 50 mg, more preferably 30 to 40mg, relating to 100 mg of the free flupirtine base. Most preferably, equimolar quantities of the free flupirtine base and the the acidic component methane sulfonate are used.

In addition, the flupirtine lyophilisate according to the invention contains one or more cyclodextrins or cyclodextrin derivatives acting as matrix forming agents. “Matrix forming agent” or “framework former” means a substance which supports the formation of a porous cake with a very big inner surface area, during and/or after lyophilisation of a given material. Hydroxyalkyl derivatives of the cyclodextrins further act as solutizers for flupirtine. In its preferred embodiment the lyophilisate according to the invention contains a Hydroxypropyl cyclodextrin as matrix forming agent. More preferably the Hydroxypropyl cyclodextrin is selected from Hydroxypropyl-α-cyclodextrin, Hydroxypropyl-β-cyclodextrin and/or Hydroxypropyl-γ-cyclodextrin. Most preferably it is Hydroxypropyl-β-cyclodextrin.

It is especially intended that the cyclodextrin(s) and/or cyclodextrin derivatives is/are contained in the lyophilisate according to the invention in a quantity of 10 mg to 1000 mg, preferably of 30 mg to 300 mg, relating to 100 mg of the free flupirtine base.

In a further preferred embodiment of the invention, it is intended that the flupirtine lyophilisate according to the invention is essentially free of antioxidants.

In the context of the present invention, a solution “essentially free of antioxidants” means that it contains less than 1 %, preferably less than 0.5 %, more preferably less than 0.1 %, and most preferably no antioxidants at all. “Antioxidants” mean excipients which may inhibit, delay or prevent the oxidation of a substance, especially that of an active ingredient.

In a further preferred embodiment of the invention, the flupirtine lyophilisate according to the invention is essentially free of detergents. In the context of the present invention, a solution “essentially free of detergents” means that it contains less than 1%, preferably less than 0.5%, more preferably less than 0.1%, and most preferably no detergents at all. Detergents here means anionic or cationic tensids or polymeric compounds with amphiphilic dissolution behaviour, such as Polyvinylpyrrolidone, which are added to reduce the surface tension of the water.

In a preferred embodiment of the invention, the pharmaceutical composition which is to be produced is a liquid pharmaceutical composition to be parenterally applied.

A dosage form or pharmaceutical composition to be “parenterally” applied means a sterile pharmaceutical composition which is applied by bypassing the gastro-intestinal tract. The advantages of parenteral application, especially compared to oral application, consist above all in rapid onset of action, avoidance of gastro-intestinal irritations, calculable blood levels of the administered active ingredient, and avoidance of the so-called first-pass effect.

Pharmaceutical compositions to be parenterally applied are, in particular, injection and infusion solutions. “Injections” or “injection solutions” are preparations of small volumes, especially containing between 1 and 20 ml, which are applied as solution, suspension, or emulsion. In an “infusion” or “infusion solution”, volumina of over 100 ml are applied.

The intravenous application permits a quick supply with, and application of, active ingredients that irritate the tissue if administered using other parenteral methods of application. Especially with regard to intramuscular and subcutaneous injections, isohydria and isotonicity need to be taken into account, as otherwise local incompatibilities may result. According to the invention it is therefore intended that the pharmaceutical composition to be parenterally applied, which is to be produced using the lyophilisate, is an injection solution or infusion solution.

The present invention likewise relates to the use of the flupirtine-containing lyophilisate according to the invention, to produce a pharmaceutical composition to be parenterally applied. According to the invention, it is here intended that the lyophilisate is used to produce the pharmaceutical composition to be parenterally applied, by dissolving the lyophilisate in an aqueous medium and/or in an organic solvent, resulting in the pharmaceutical composition to be parenterally applied.

As an aqueous medium according to the invention, preferably water is used, more preferably water for injection. In a different embodiment of the invention it is intended that a suitable isotonic solution, preferably an isotonic sodium chloride solution, is used as an aqueous medium.

The present invention likewise relates to a procedure to produce a flupirtine-containing pharmaceutical composition to be parenterally applied, where a flupirtine-containing lyophilisate according to the invention is dissolved in an aqueous medium and/or in an organic solvent resulting in a ready-to-use liquid pharmaceutical composition.

Preferably, the flupirtine lyophilisate according to the invention is dissolved at room temperature. In an especially preferred embodiment of the invention, the flupirtine lyophilisate according to the invention is dissolved in water, especially in water for injection. However, the flupirtine lyophilisate may also be dissolved in a buffer solution, in an isotonic sodium chloride solution, or in a mixture of water and solvent.

The isotonicity of the resulting solution may be adjusted through the volume of the aqueous medium used to dissolve the lyophilisate. Before application of the pharmaceutical composition to be parenterally applied, it may be decided how the lyophilisate is to be reconstituted, and whether it may be diluted, if necessary. Thus, the lyophilisate according to the invention allows the preparation of both an intramuscular and an intravenous injection. The reconstituted aqueous preparation may also be used as an admixture in current infusion solutions.

According to the invention it is therefore intended that the pharmaceutical composition to be parenterally applied, which has been produced using said procedure, is an injection solution. If the injection solution to be produced is to be applied intravenously, it is intended according to the invention that the lyophilisate according to the invention, which preferably contains 50to 250 mg of flupirtine, is dissolved in 1 to 20 ml, preferably in 3 to 5 ml of water for injection, buffer solution, or a mixture of water and solvent.

If the injection solution to be produced is to be applied intramuscularly, it is intended according to the invention, that the lyophilisate according to the invention, which preferably contains 50 to 250 mg of flupirtine, more preferably 80 to 120 mg of flupirtine, is dissolved in 3 ml of water for injection, buffer solution, or a mixture of water and solvent. In a further preferred embodiment of the invention it is intended that the pharmaceutical composition to be parenterally applied is an injection solution.

The present invention likewise relates to a procedure to produce the flupirtine-containing lyophilisate according to the invention, comprising
a) dissolving at least one cyclodextrin and/or cyclodextrin derivative in an aqueous solution and/or an organic solvent,
b) introducing an acid which forms a physiologically tolerated salt with the flupirtine base into the cyclodextrin solution respectively cyclodextrin derivative solution,
c) adding the flupirtine base,
d) heating and cooling the solution,
e) if necessary, diluting the solution,
f) if necessary, adjusting its pH value and its tonicity,
g) freeze-drying the resulting flupirtine solution.

According to the invention, it is therefore intended that in the first step the cyclodextrin(s) and/or cyclodextrin derivative(s) is/are dissolved in an aqueous solution. After that, the acid is added to the resulting solution and mixed; in the next step flupirtine is added and with this, the acid forms the physiologically tolerated flupirtine salt.

Preferably, this flupirtine salt is one which has a solubility in water of at least 2.5 mg/ml. Preferably its solubility in water is at least 5 mg/ml, more preferably at least 10 mg/ml.

In an especially preferred embodiment, the acid is selected from methanesulfonic acid and benzene sulfonic acid. Most preferably the acid is methanesulfonic acid.

After that, the flupirtine base, preferably in solid form and in portions, is introduced to the solution while stirring. The mixture is heated, preferably to a temperature between 70 and 90° C., preferably between 75 and 85° C., and after reaching this temperature it is cooled again to 25° C.

In one embodiment of the method according to the invention aforementioned steps b) and c) are replaced by a step of introducing the respective flupirtine salt, preferably flupirtine mesylate, into the solution obtained by step a).

The obtained clear solution is adjusted, preferably with an aqueous solution, to the desired final volume.

Der pH value may be adjusted using a suitable base, preferably NaOH, KOH, ammonia, but also soluble carbonates, to the desired pH value. The composition according to the invention may be adjusted to a pH value between 3.0 and 3.9, preferably between 3.5 and 3.7, without impairing the stability of the composition.

In addition, the tonicity of the solution can be increased, if necessary, by adding NaCl. Isotonic solutions have about the same osmolarity (300 mosm/L) as the blood plasma, if the dissolved substances are unable to freely permeate the cell walls.

In a further preferred embodiment the solution is filtered before freeze-drying. Afterwards, the preferably filtered solution containing flupirtine salts is filled into freeze-drying flasks which are then provided with freeze-drying stoppers. To freeze the solution containing flupirtine salts, the freeze-drying flasks are stored at −50° C. to −40° C.

According to the invention, it is intended that that the actual freeze drying consists of main drying and secondary drying. In the preferred embodiment the main drying takes place at a temperature of −50° C. to −15 ° C. and at a pressure of 0.050 to 0.150 mbar. In the preferred embodiment, afterwards the secondary drying takes place at a temperature of 15° C. to 45° C. and at a pressure of 0.001 to 0.010 mbar. The flasks containing the flupirtine lyophilisate are then closed sterilely, preferably under a nitrogen atmosphere.

The present invention likewise relates to the liquid pharmaceutical composition to be parenterally applied which may be obtained by reconstituting the flupirtine lyophilisate according to the invention.

The present invention is explained in more detail by the following examples.

### EXAMPLE 1

### Production of a Flupirtine-Containing Lyophilisate-1

In a vessel containing 850 ml water for injection, 83.3 g of Hydroxypropyl-β-cyclodextrin is added while stirring, and after it has dissolved, 10.5 g of methanesulfonic acid is added. 33.33 g of flupirtine base is added by portions while stirring. At first, this results in a clear solution, in which a precipitate forms shortly afterwards. The mixture is heated to 80° C., and at 50° C. a clear solution forms again. After reaching 80° C., it is cooled to 25° C. The clear solution is adjusted to pH 3.5 using 10 % NaOH and filled up with water for injection to the final volume of 1000 ml. The solution is filtered through a 0.2 urn filter. For freeze-drying, 3.0 ml at a time is filled into 10 R vials. Freeze drying took place at a temperature of −30° C. and 0.140 mbar, the final drying at +30° C. and 0.007 mbar. This resulted in a visually very good lyophilisate, which dissolves rapidly after adding 3.0 ml of water for injection. The reconstituted solution was physically and chemically stable over 12 hours.

### Composition

One vial contains:

| | | |
|---|---|---|
| 100.00 | mg | flupirtine |
| 250.00 | mg | hydroxypropyl-β-cyclodextrin |
| 31.58 | mg | methanesulfonic acid |
| approx. 0.4 | mg | sodium hydroxide |

### EXAMPLE 2

### Production of a Flupirtine-Containing Lyophilisate-1

A vessel containing 850 ml water for injection is heated to 50° C. While stirring, 83.3 g of Hydroxypropyl-β-cyclodextrin is added. After it has dissolved, 41.87 g flupirtine mesylate is added in small portions at a temperature of 50° C. and while stirring the solution. A clear solution is formed that is heated to 80° C. After reaching the temperature of 80° C. the solution is cooled down to 25° C. The clear solution is adjusted to pH 3.5 using 10% NaOH and filled up with water for injection to the final volume of 1000 ml. The solution is filtered through a 0.2 μm filter. For freeze-drying, 3.0 ml at a time is filled into 10 R vials. Freeze drying took place at a temperature of −30° C. and 0.140 mbar, the final drying at +30° C. and 0.007 mbar. This resulted in a visually very good lyophilisate, which dissolves rapidly after adding 3.0 ml of water for injection. The reconstituted solution was physically and chemically stable over 12 hours.

### Composition

One vial contains:

| | | |
|---|---|---|
| 125.63 | mg | flupirtine mesylate (corresponding |
| | | to 100.00 mg flupirtine) |
| 250.00 | mg | hydroxypropyl-β-cyclodextrin |
| approx. 0.4 | mg | sodium hydroxide |

### EXAMPLE 3

### Production of a Flupirtine-Containing Lyophilisate-2

To the solution filled up to 1000 ml, produced as in Example 1 or 2, 3 g of sodium chloride is added. The solution is filtered through a 0.2 μm filter. For freeze-drying, 3.0 ml at a time is filled into 10 R vials. Freeze drying took place at −30° C. and 0.140 mbar, the final drying at +30° C. and 0.007 mbar. This resulted in a visually very good lyophilisate, which dissolves rapidly after adding 3.0 ml water for injection. The reconstituted solution was physically and chemically stable over at least 12 hours.

### Composition

One vial produced as in Example 1 contains:

| | | |
|---|---|---|
| 100.00 | mg | flupirtine |
| 250.00 | mg | hydroxypropyl-β-cyclodextrin |
| 31.58 | mg | methanesulfonic acid |
| approx. 0.4 | mg | sodium hydroxide |
| 9.0 | mg | sodium chloride |

One vial produced as in Example 2 contains:

| | | |
|---|---|---|
| 125.63 | mg | flupirtine mesylate (corresponding |
| | | to 100.00 mg flupirtine) |
| 250.00 | mg | hydroxypropyl-β-cyclodextrin |
| approx. 0.4 | mg | sodium hydroxide |
| 9.0 | mg | sodium chloride |

### EXAMPLE 4

### Production of a Liquid Pharmaceutical Composition

3 ml of water for injection is added to one bottle of the lyophilisate described in Example 1. After occasional swirling, a clear solution is obtained after about 1 min. The slightly hypotonic solution may be directly applied intravenously.

### EXAMPLE 5

### Production of a Liquid Pharmaceutical Composition

3 ml of isotonic sodium chloride solution is added to one bottle of the lyophilisate described in Example 1. After occasional swirling, a clear solution is obtained after about 1 min. The slightly hypertonic solution may be directly applied intramuscularly.

What is claimed is:

1. A lyophilisate, which contains a physiologically tolerated flupirtine sail with a solubility in water of at least 2.5 mg/ml as an active ingredient and furhter contains at least one cyclodextrin and/or cyclodextrin derivative.

2. The lyophilisate according to claim 1, containing 50 mg to 250 mg flupirtine, relating to the free flupirtine base.

3. The lyophilisate according to claim 1, wherein the flupirtine salt is selected from the group consisting of flupirtine mesilate, flupirtine besilate and flupirtine phosphate.

4. The lyophilisate according to claim 1, wherein the flupirtine salt is flupirtine mesilate.

5. The lyophilisate according to claim 4, further containing methanesulfonic acid in a quantity of 10 mg to 100 mg, preferably of 20 mg to 50 mg, especially preferably of 30 mg to 40 mg, relating to 100 mg of the free flupirtine base.

6. The lyophilisate according to claim 1, wherein the cyclodextrin derivative is selected from the group consisting of hydroxypropyl-α-cyclodextrin, hydroxypropyl-β-cyclodextrin and hydroxypropyl-γ-cyclodextrin.

7. The lyophilisate according to claim 1 wherein the cyclodextrin and/or cyclodextrin derivative is/are contained in a total quantity of 10 mg to 1000 mg, preferably of 30 mg to 300 mg, relating to 100 mg of the free flupirtine base.

8. The pharmaceutical composition according to claim 9, wherein the pharmaceutical composition to be parenterally applied is an injection solution and/or infusion solution.

9. A pharmaceutical composition to be parenterally applied containing a lyophilisate according to claim 1.

10. The pharmaceutical composition according to claim 9, obtained by dissolving the lyophilisate in an aqueous medium and/or in an organic solvent.

11. A procedure to produce a lyophilisate according to claim 1, comprising the steps:
a) dissolving one or more cyclodextrins and/or cyclodextrin derivatives in an aqueous medium and/or an organic solvent,
b) introducing an acid which forms a physiologically tolerated salt with flupirtine base into the solution prepared in step a),
c) adding flupirtine base to the solution of step b),
d) heating and cooling the solution of step c),
e) if necessary, diluting the solution of step c),
f) if necessary, adjusting a pH value and a tonicity of the solution of step c),
g) freeze-drying the solution of step c).

12. The procedure according to claim 11, wherein the aqueous medium used for dissolving the flupirtine base is healed in step d) to a temperature above room temperature and subsequently cooled to room temperature.

13. A procedure to produce a flupirtine-containing pharmaceutical composition to be parenterally applied, wherein a lyophilisate according to claim 1 is dissolved in an aqueous medium and/or an organic solvent and a ready-to-use liquid pharmaceutical composition is obtained,

14. The pharmaceutical composition according to claim 10, in the form of an injection solution to be applied intravenously and/or intramuscularly.

15. A liquid flupirtine-containing pharmaceutical composition to be parenterally applied, produced by dissolving a lyophilisate according to claim 1.
